(19) United States
(12) Patent Application Publication
(10) Pub. No.: US 2015/0352159 A1
(43) Pub. Date: Dec. 10, 2015

(54) Method and preparation for treating arthritis and arthrosis

(71) Applicant: (OBSCHESTVO S OGRANICHENNOJ OTVETSTVENNOSTJU "PARAFARM"), Penza (RU)

(72) Inventors: Villorij I. Strukov, Penza (RU); Mikhail D. Prokhorov, Penza (RU); Olga Jones-Strukova, Grand Prairie, TX (US); Vjacheslav N. Trifonov, Zarechny, Penzenskaya obl. (RU); Julija A. Elistratova, Penza (RU); Konstantin G. Elistratov, Penza (RU); Natal'ja V. Kurus', Penza (RU); Natalya V. Eremina, Penza (RU); Marina N. Maksimova, Penza (RU); Ramziya T. Galeeva, Penza (RU); Larisa G. Radchenko, Penza (RU); Aleksandr V. Fedorov, Kuznetsk, Penzenskaya obl. (RU); Evgeny N. Krutyakov, Penza (RU); Elena S. Andreeva, Penza (RU); Tatiana V. Elistratova, Penza (RU); Irina V. Khomyakova, Penza (RU); Galina A. Tolbina, Penza (RU)

(73) Assignee: Parafarm LLC, Penza (RU)

(21) Appl. No.: 14/760,702

(22) PCT Filed: Jan. 13, 2014
(86) PCT No.: PCT/RU2014/000009

§ 371 Date: Jul. 13, 2015

(30) Foreign Application Priority Data
Jan. 11, 2013 (RU) 2013 100994

(51) Int. Cl. A61K 35/64; A61K 33/10; A61K 33/06

(52) U.S. Cl. CPC A61K 35/64; A61K 33/06; A61K 33/10

(57) ABSTRACT

The present method and preparation for treating arthritis and arthrosis relates to medicine, and more particularly to methods for controlling arthritis and arthrosis. The method is implemented by filling cavity lesions in the bone tissue and consists in providing to the body a supply of drone brood in conjunction with a supply of calcium.

Invention relates to medicine, and particularly, to methods of treatment of arthritis and osteoarthrosis.

There is known a phytotherapy medicament “Alevalon” for treatment of arthritis and method of its preparation (2102998, Feb. 10, 1995). A medicament for treatment of arthritis and method of its preparation relate to compositions which contain substances from plants, and can be used as a base for remedy for treatment of mono- and polyarthritis, and osteoarthrosis. The composition comprises ethanolic extract of following ingredients in following ratio (in parts by weight): roots of Inula helenium—2-5 parts, roots of Comarum palustre—1-4 parts, buds of birch (Betula), the rest—ethanol. The composition can also include ethanolic extract of pantocrine or ginseng. The method comprises alternate introduction of the above mentioned components in solution, including its heating and periodic shaking.

The main disadvantages of this known composition and method of its preparations are that they in common are aimed to the treatment of arthritis and don't concern the problem of osteoarthrosis.

The closest to the above mentioned technical solution is method of treatment of rheumatoid arthritis (Patent of Russian Federation Nr. 2268734, Jun. 21, 2004). Invention relates to medicine, and particularly, to rheumatology, and aimed to treatment of any forms and phases of rheumatoid arthritis (RA). Method includes base immunomodulatory and symptomatic therapies, and, additionally, treatment by composition Calcium-D3 with adjustment of dose, duration, and multiplicity of treatment courses depending the phase, degree, characteristics of course of disease, and bone mineral density (BND).

The main disadvantage of the above mentioned method of treatment is the high risk of development of hypercalciemia, as well as that this method is not aimed to the treatment of osteoarthrosis. The more detailed description of treatment of hypercalciemic states gives Prof. V. I. Strukov in his book “Disorders of calcium metabolism. Hypercalciemic states”/Ed. by Penza Institute of physician's postgraduate training. Penza, “Rodnoi dom”, 2011.

Technical result to which is aimed claimed invention is to obtain the closing of cavities in bony tissue and to accelerate the recovery of patients having arthritis and osteoarthrosis.

This technical result is being obtained by administering of drone brood and calcium compound. According to the invention, drone brood and calcium compound must be taken in simultaneously during 24 hours. Calcium compound and drone brood for treatment of arthritis and osteoarthrosis may be in form of form of powder, tablets or capsules, wherein calcium compound is one from the following group of compounds or any combination of them—calcium carbonate, calcium citrate, calcium gluconate, calcium aspartate, calcium ascorbate, calcium aminochelate, calcium fumarate, calcium succinate, and calcium phosphate.

Osteoarthrosis and arthritis, internationally known as osteoarthritis, are widespread group of diseases of various origins, for which are characteristic development of dystrophic and inflammatory changes in bony tissue.

The most widespread today in CIS countries are some dangerous and inefficient programs of treatment of “polyarthritis” with steroid and non-steroid anti-inflammatory and analgetic medicaments which assure only symptomatic effect. These methods have a series of important disadvantages because they don't pay attention to age-related causes of arthritis and osteoarthrosis.

We have ascertained that the cause of age-related osteoarthrosis and/or arthritis is presence of cavities in bony tissue. As a result, process of building of bony tissue is being felt behind the process of its destruction by osteoclasts.

Formed thereby bone cavities worsen normal metabolism of that bone part which is located behind cavity. As a result, when osteoclast destroys a bone cell, bone mineral matrix can do with it nothing, since the bone cavity do not allow it to be excreted from bony tissue. Therefore, the bone mineral matrix is excreted by organism from the bone. The result of its process is osteoarthrosis. Thus, the cause of osteoarthrosis is not consequence of age-related changes in muscle frame or way of stabilizing of human skeleton. The cause of osteoarthrosis consists in bone cavities which are hampering excretion of destroyed bone mineral matrix from the bones.

The cause of development of arthritis is the same: inflammatory processes in bony tissue. The bone cavity hampers the excretion of waste products of bone cells and destroyed cells from bony tissue. It results in inflammation—arthritis.

We have established during treatment of osteoporosis by “Osteomed” supplement that the use of drone brood allows closing of bone cavities. As soon as bone cavities begin to close, arthritic and osteoartotic processes also begin to stop and to turn backwards.

The speed of closing of bone cavities depends on the degree of calcium absorption by organism and may be increased with sufficient intake of calcium.

We have also established that even one-month administering of drone brood, plus diet with a great amount of calcium, gives a positive results: in patients treated with these products are disappeared aching pains in bones that allowed them to give up the use of non-steroid anti-inflammatory remedies.

Further administering of the claimed composition leads to more closing of bone cavities thereby improving metabolism of bony tissue located behind bone cavity. Progression of the disease stops, joint mobility improves, pains during motion decrease. The practice of using of the claimed composition in more than 1000 patients with osteoarthrosis and arthritis demonstrated significant progress in therapy after 6-9 months of treatment. Patients which earlier could not move because of severe pains began to walk and serve themselves.

During experimental confirmation drone brood was administered to patients orally in form of powder, tablets or capsules, both as separate acting agent as well as in combination with calcium compounds.

Depending on individual features of organism, amount of calcium taken with food, quantity and dimensions of bone cavities, the positive result may be achieved faster or slower, but it was obtained in all cases. As showed the practice, the practically significant results were obtained after long enough administering (9 months) in 100% of patients.

1. A method of treatment of arthritis and osteoarthrosis which includes intake of drone brood and calcium compounds.

2. The method of claim 1 wherein the drone brood and calcium compounds are taken in simultaneously during 24 hours.

3. A composition for treatment of arthritis and osteoarthrosis which comprises drone brood and calcium compounds.

4. The composition of claim 3 wherein it is made a form of powder, tablets or capsules.

5. The composition of claim 3 wherein the calcium compound is one from the following group of compounds or any combination of them—calcium carbonate, calcium citrate, calcium gluconate, calcium aspartate, calcium ascorbate, calcium aminochelate, calcium fumarate, calcium succinate, and calcium phosphate.
